(19) United States
(12) Patent Application Publication
(10) Pub. No.: US 2014/0328893 A1
(43) Pub. Date: Nov. 6, 2014

(54) NUTLIN COMPOUNDS FOR USE IN THE TREATMENT OF PULMONARY HYPERTENSION

(71) Applicant: Serge Adnot, Creteil (FR)

(72) Inventor: Serge Adnot, Creteil (FR)

(73) Assignee: Institut National de la Sante et de la Recherche Medicale (INSERM), Paris (FR)

(21) Appl. No.: 14/350,910

(22) PCT Filed: Oct. 11, 2011
(86) PCT No.: PCT/IB2011/002832

§ 371 Date: Jun. 17, 2014

(51) Int. Cl. A61K 31/496; A61L 31/16; A61L 29/16; A61K 45/06

(52) U.S. Cl. CPC A61K 31/496; A61K 45/06; A61L 31/16; A61L 29/16
USPC 424/423; 514/254.05; 544/370; 424/430

(57) ABSTRACT

The present invention relates to uses, methods and compositions for the treatment of pulmonary hypertension.

### FIELD OF THE INVENTION

The present invention relates to uses, methods and compositions for the treatment of pulmonary hypertension.

### BACKGROUND OF THE INVENTION

Pulmonary hypertension (PH) is a fatal disease caused by small pulmonary artery obstruction from vascular proliferation and remodeling. PH is characterized by elevated pulmonary arterial pressure and increased pulmonary vascular resistance, frequently leading to right-sided heart failure and death (Fukumoto and Shimokawa, 2011).

Activation of the p53 tumor suppressor protein may be useful for inducing pulmonary-artery smooth muscle cell (PA-SMC) senescence or apoptosis during the course of pulmonary hypertension (PH). Nutlins are cis-imidazoline analogues that antagonize the interaction between p53 and MDM2 (murine double minute 2), thereby directly stabilizing p53 by preventing its proteosomal degradation.

There is no disclosure in the art of the use of nutlin compound in methods for inhibiting pulmonaty-artery smooth muscle cell proliferation, nor the use of nutlin compound in methods for inducing pulmonary-artery smooth muscle cell senescence or apoptosis, nor the use of nutlin compound in methods for treatment of pulmonary hypertension.

### SUMMARY OF THE INVENTION

The first object of the invention relates to a nutlin compound for use in the prevention or treatment of pulmonary hypertension in a subject of need thereof.

Another object of the invention relates to a nutlin compound for use in a method for inhibiting pulmonary-artery smooth muscle cell proliferation in a subject afflicted with pulmonary hypertension.

Another object of the invention relates to a nutlin compound for use in a method for inducing pulmonary-artery smooth muscle cell senescence or apoptosis in a subject afflicted with pulmonary hypertension.

Another object of the invention relates to a nutlin compound for use in a method for inducing pulmonary-artery smooth muscle cell senescence or apoptosis and/or inhibiting pulmonary-artery smooth muscle cell proliferation in a subject of need thereof.

### DETAILED DESCRIPTION OF THE INVENTION

The role of nutlin in pulmonary hypertension was investigated by the inventors using transgenic mice expressing the human 5-HTT gene in smooth muscle (SM22-5-HTT+ mice), p53-deficient mice (p53−/− mice), p21-deficient mice (p21−/− mice), pulmonary hypertension induction by exposure to chronic hypoxia and cultured human pulmonary artery smooth muscle cells (PA-SMC). The inventors surprisingly found that Nutlin-3 is involved in inhibition of PA-SMC proliferation and PH development in mice exposed to chronic hypoxia and in transgenic mice overexpressing the serotonin transporter in SMCs (SM22-5-HTT+ mice). The inventors also demonstrate that hypoxic PH development and treatment with nutlin were altered in p53-deficient (p53^{-/-}) mice and in p21-deficient (p21−/−) mice. Thus, the protective effect mediated by nutlin required expression of p53 and p21 in the lung. The inventors also demonstrate that nutlin is involved in induction of pulmonary-artery smooth muscle cell senescence and apoptosis and in activation of p53 pathway and thereby may lead to the treatment of pulmonary hypertension.

Accordingly the present invention relates to a nutlin compound for use in the prevention or treatment of pulmonary hypertension in a subject of need thereof.

The term “nutlin” has its general meaning in the art and refers to nutlin analogues or cis-imidazoline analogues.

The method of the invention may be performed for any type of pulmonary hypertension such as revised in the World Health Organisation Classification of pulmonary hypertension and selected from the group consisting of Pulmonary arterial hypertension that develops as sporadic disease (idiopathic), as an inherited disorder (familial), or in association with certain conditions (collagen vascular diseases, congenital systemic-to-pulmonary shunts (large, small, repaired, or nonrepaired), portal hypertension, human immunodeficiency virus (HIV) infection, ingestion of drugs or dietary products and toxins (anorexigens, rapeseed oil, L-tryptophan, methamphetamine, and cocaine), or in association with other conditions (thyroid disorders, glycogen storage disease, Gaucher disease, hereditary hemorrhagic telangiectasia, hemoglobinopathies, myeloproliferative disorders, and splenectomy)), or associated with significant venous or capillary involvement (pulmonary veno-occlusive disease and pulmonary capillary hemangiomatosis) and persistent pulmonary hypertension of the newborn; Pulmonary venous hypertension (left-sided atrial or ventricular heart disease, left-sided valvular heart disease); Pulmonary hypertension associated with hypoxemia (chronic obstructive pulmonary disease, Interstitial lung disease; Sleep-disordered breathing, alveolar hypoventilation disorders, Long-term exposure to high altitudes, developmental abnormalities); Pulmonary hypertension due to chronic thrombotic or embolic disease (thromboembolic obstruction of proximal pulmonary arteries, thromboembolic obstruction of distal pulmonary arteries, Pulmonary embolism (tumor, parasites, foreign material)); Miscellaneous (sarcoidosis, pulmonary Langerhans'-cell histiocytosis, lymphangiomatosis, compression of pulmonary vessels (adenopathy, tumor, fibrosing mediastinitis) (Raiesdana and Loscalzo, 2006; Simonneau et al., 2004).

As used herein, the term “subject” denotes a mammal. In a preferred embodiment of the invention, a subject according to the invention refers to any subject (preferably human) afflicted with pulmonary hypertension. In a particular embodiment, the subject is afflicted with pulmonary arterial hypertension. In a particular embodiment, the subject is afflicted with pulmonary arterial hypertension associated HIV infection.

The term “nutlin compound” has its general meaning in the art and refers to cis-imidazoline analogues described by Vassilev et al., 2004.

Said nutlin compounds are well known in the state of the art as illustrated by Deng et al., 2006; US2005/0288287 and WO2010/028858.

In a particular embodiment, the compound according to the invention may be nutlin compounds that are the representatives of a class of cis-2,4,5-triphenyl-imidazolines (see for example, Vassilev et al., 2004; Vassilev, 2005; Klein and Vassilev, 2004; WO2003/051359; US2004/0204410; US2003/0153580).

In a particular embodiment, the compound according to the invention may be a cis-imidazoline analogue (see for example US2004/0259884; WO2005/003097; US2004/0259867; WO2005/002575).

In one embodiment of the invention, the nutlin compound is selected in the group consisting of nutlin 1, nutlin 2, nutlin 3 ((±)-4-[4,5-Bis(4-chlorophenyl)-2-(2-isopropoxy-4-methoxy-phenyl)-4,5-dihydro-imidazole-1-carbonyl]-piperazin-2-one), nutlin 3a and pharmaceutically acceptable salts, esters, solvates or derivates of the above.

Another object of the invention relates to a nutlin compound for use in a method for inhibiting pulmonary-artery smooth muscle cell proliferation in a subject afflicted with pulmonary hypertension.

Another object of the invention relates to a nutlin compound for use in a method for inducing pulmonary-artery smooth muscle cell senescence or apoptosis in a subject afflicted with pulmonary hypertension.

Another object of the invention relates to a nutlin compound for use in a method for inducing pulmonary-artery smooth muscle cell senescence or apoptosis and/or inhibiting pulmonary-artery smooth muscle cell proliferation in a subject of need thereof.

Another object of the invention relates to a pharmaceutical composition comprising a nutlin compound and a pharmaceutically acceptable carrier for use in the prevention or treatment of pulmonary hypertension in a subject in need thereof.

Another object of the invention relates to a pharmaceutical composition comprising a nutlin compound and a pharmaceutically acceptable carrier for use in a method for inhibiting pulmonary-artery smooth muscle cell proliferation in a subject afflicted with pulmonary hypertension.

Another object of the invention relates to a pharmaceutical composition comprising a nutlin compound and a pharmaceutically acceptable carrier for use in a method for inducing pulmonary-artery smooth muscle cell senescence or apoptosis in a subject afflicted with pulmonary hypertension.

Another object of the invention relates to a pharmaceutical composition comprising a nutlin compound and a pharmaceutically acceptable carrier for use in a method for inducing pulmonary-artery smooth muscle cell senescence or apoptosis and/or inhibiting pulmonary-artery smooth muscle cell proliferation in a subject of need thereof.

Typically, nutlin compound may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The nutlin compound can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further agent which is used in the prevention or treatment of pulmonary hypertension. For example, the anti-pulmonary hypertension may include supplemental oxygen, diuretics, anticoagulants, calcium-channel blockers such as intravenous epoprostenol, adenosine, nifedipine, diltiazem, amlodipine, or inhaled nitric oxide, prostanoids such as prostacyclin derivatives, epoprostenol, treprostinil, iloprost, treprostinil, beraprost, prostaglandins, prostacyclin (prostaglandin I2), endothelin receptor antagonists such as bosentan, Sitaxsentan, ambrisentan and Actelion-1, nitric oxide and phosphodiesterase-5 inhibitors such as sildenafil and tadalafil. Said anti-pulmonary hypertension may include beta blockers, angiotensin-converting enzyme (ACE) inhibitors, digoxin, adenosine, vasoactive substances, guanylate cyclase activators (sGC), cinaciguat, riociguat, 5′-adenosine monophosphate-activated protein kinase (AMPK) activators, rho-kinase inhibitors, serotonin antagonists, phosphodiesterase-1 inhibitors, vasoactive intestinal peptide (VIP) and cyclophosphamide (Raiesdana and Loscalzo, 2006; Fukumoto and Shimokawa, 2011; Fuso et al., 2011).

Pharmaceutical compositions of the invention may include any further agent which is used in the induction of pulmonary-artery smooth muscle cell senescence or apoptosis, the activation and/or stabilization of p53, the inhibition and/or antagonizing p53 and MDM2 interaction, the activation and/or increasing the expression of p53, p21, Bax and PUMA, the prevention or treatment of lung inflammation, the prevention or treatment of vascular remodelling, the inhibition of lung inflammation cell infiltrates or inhibition of cytokine expression.

In one embodiment, said additional agents may include but are not limited to benzodiazepine derivates, piperazine derivates, piperidine derivates, aryl boronic acids, fused indoles, spiro-oxindoles, α-helix mimetic compounds.

In one embodiment, said additional active agents may be contained in the same composition or administrated separately.

In another embodiment, the pharmaceutical composition of the invention relates to combined preparation for simultaneous, separate or sequential use in the treatment of pulmonary hypertension.

Pharmaceutical compositions of the invention may include any further agent which has the capacity of limiting the cyclic nucleotides (cGMP, cAMP) elimination. Such agents may include but are not limited to specific phosphodiesterase (PDE) superfamily inhibitors including PDE3, PDE4 and PDE5 inhibitors. Examples of PDE4 inhibitors include rolipram and those described in patent documents US2005234238 DE10156229, DE10135009 and WO0146151. Examples of PDE5 inhibitors include sildenafil, vardenafil and tadalafil. Particularly preferred are PDE5 inhibitors that are marketed, e.g. VIAGRA(R) which is sildenafil citrate and which can be administered in this form. Other examples of PDE5 inhibitors also include those described in patent documents WO2005012303 and US2006106039.

Pharmaceutical compositions of the inventions may include any other anti-proliferative agent that reduce smooth muscle cell proliferation. For example, the anti-proliferative agent may be rapamycin, rapamycin derivatives, paclitaxel, docetaxel, 40-0-(3-hydroxy)propyl-rapamycin, 40-O-[2-(2-hydroxy)ethoxy]ethyl-rapamycin, and 40-O-tetrazole-rapamycin, ABT-578, everolimus and combinations thereof.

Pharmaceutical compositions of the invention may include compounds selected from the group consisting of antibodies, receptor ligands, enzymes, adhesion peptides, oligosaccharides, oligonucleotides and the like. Such compounds may be blood clotting factors, inhibitors or clot dissolving agents such as streptokinase and tissue plasminogen activator. Such agents can also include a prohealing drug that imparts a benign neointimal response characterized by controlled proliferation of smooth muscle cells and controlled deposition of extracellular matrix with complete luminal coverage by phenotypically functional (similar to uninjured, healthy intima) and morphologically normal (similar to uninjured, healthy intima) endothelial cells. Such compounds can also fall under the genus of antineoplastic, cytostatic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL(R) by Bristol-Myers Squibb Co., Stamford, Conn.), docetaxel (e.g. Taxotere(R), from Aventis S. A., Frankfurt, Germany) methotrexate, azathioprine, vincristine, vinblastine, fluorouracil, doxorubicin hydrochloride (e.g. Adriamycin(R) from Pharmacia & Upjohn, Peapack N.J.), and mitomycin (e.g. Mutamycin(R) from Bristol-Myers Squibb Co., Stamford, Conn.). Examples of such antiplatelets, anticoagulants, antifibrin, and antitlirombins include heparinoids, hirudin, recombinant hirudin, argatroban, forskolin, vapiprost, prostacyclin and prostacyclin analogues, dextran, D-phe-pro-arg-chloromethylketone (synthetic antithrombin), dipyridamole, glycoprotein Ilb/Illa platelet membrane receptor antagonist, antibody, and thrombin inhibitors such as Angiomax(R) (Biogen, Inc., Cambridge, Mass.). Examples of cytostatic agents include angiopeptin, angiotensin converting enzyme inhibitors such as captopril (e.g. Capoten and Capozide(R) from Bristol-Myers Squibb Co., Stamford, Conn.), cilazapril or lisinopril (e.g. Prinivil(R) and Prinzide(R) from Merck & Co., Inc., Whitehouse Station, N.J.), actinomycin D, or derivatives and analogs thereof. Synonyms of actinomycin D include dactinomycin, actinomycin IV, actinomycin I1, actinomycin X1, and actinomycin Ci. Other compounds include calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor(R) from Merck & Co., Inc., Whitehouse Station, N.J.), monoclonal antibodies (such as those specific for Platelet-Derived Growth Factor (PDGF) receptors), nitroprusside, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, genetically engineered epithelial cells, antibodies such as CD-34 antibody, abciximab (REOPRO), and progenitor cell capturing antibody, prohealing drugs that promotes controlled proliferation of muscle cells with a normal and physiologically benign composition and synthesis products, enzymes, anti-inflammatory agents, antivirals, anticancer drugs, anticoagulant agents, free radical scavengers, steroidal anti-inflammatory agents, non-steroidal anti-inflammatory agents, antibiotics, nitric oxide donors, super oxide dismutases, super oxide dismutases mimics, 4-amino-2,2,6,6-tetramethylpiperidine-1-oxyl (4-amino-TEMPO), dexamethasone, clobetasol, aspirin, prodrugs thereof, co-drugs thereof, and a combination thereof. The foregoing substances are listed by way of example and are not meant to be limiting. Other active agents which are currently available or that may be developed in the future are equally applicable.

The present invention also relates to a kit for treating a pulmonary hypertension comprising a first pharmaceutical composition comprising a nutlin compound and a second pharmaceutical composition comprising one or more Phosphodiesterase (PDE) inhibitors selected from the group consisting of PDE3 inhibitors, PDE4 inhibitors, PDE5 inhibitors and mixtures thereof.

Another object of the invention relates to a pharmaceutical composition according to the invention comprising one or more chemotherapeutic or radiotherapeutic agents.

In one embodiment said chemotherapeutic or radiotherapeutic agents are a therapeutic active agent used as anticancer agent. For example, said anticancer agents include but are not limited to fludarabine, gemcitabine, capecitabine, methotrexate, taxol, taxotere, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, anthracyclines, MDR inhibitors and Ca²⁺ ATPase inhibitors.

Additional anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anticancer agent may be selected from, but are not limited to, growth or hematopoietic factors such as erythropoietin and thrombopoietin, and growth factor mimetics thereof.

Further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

A further object of the invention relates to kits for performing the methods of the invention, wherein said kits comprise a nutlin compound for use in the prevention or treatment of pulmonary hypertension in a subject of need thereof.

A further object of the invention relates to kits comprising a nutlin compound for use in a method for inhibiting pulmonary-artery smooth muscle cell proliferation in a subject afflicted whith pulmonary hypertension.

A further object of the invention relates to kits comprising a nutlin compound for use in a method for inducing pulmonary-artery smooth muscle cell senescence or apoptosis in a subject afflicted with pulmonary hypertension.

A further object of the invention relates to kits comprising a nutlin compound for use in a method for inducing pulmonary-artery smooth muscle cell senescence or apoptosis and/or inhibiting pulmonary-artery smooth muscle cell proliferation in a subject of need thereof.

A further object of the invention relates to kits comprising a pharmaceutical composition according to the invention and a pharmaceutically acceptable carrier for use in the prevention or treatment of pulmonary hypertension in a subject in need thereof.

The present invention also relates to the use of a nutlin compound for the preparation of biomaterials or medical delivery devices selected among endovascular prostheses, such as stents, bypass grafts, internal patches around the vascular tube, external patches around the vascular tube, vascular cuff, and angioplasty catheter.

In this respect, the invention relates more particularly to biomaterials or medical delivery devices as mentioned above, coated with such nutlin compound as defined above, said biomaterials or medical devices being selected among endovascular prostheses, such as stents, bypass grafts, internal patches around the vascular tube, external patches around the vascular tube, vascular cuff, and angioplasty catheter. Such a local biomaterial or medical delivery device can be used to reduce stenosis as an adjunct to revascularizing, bypass or grafting procedures performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoro-ethylene grafting and with or without stenting, or in conjunction with any other heart or transplantation procedures, or congenital vascular interventions.

For illustration purpose, such endovascular prostheses and methods for coating nutlin compound thereto are more particularly described in WO2005094916, or are those currently used in the art. The compounds used for the coating of the prostheses should preferentially permit a controlled release of said inhibitor. Said compounds could be polymers (such as sutures, polycarbonate, Hydron, and Elvax), biopolymers/biomatrices (such as alginate, fucans, collagen-based matrices, heparan sulfate) or synthetic compounds such as synthetic heparan sulfate-like molecules or combinations thereof. Other xamples of polymeric materials may include biocompatible degradable materials, e.g. lactone-based polyesters orcopolyesters, e.g. polylactide; polylactide-glycolide; polycaprolactone-glycolide; polyorthoesters; polyanhydrides; polyaminoacids; polysaccharides; polyphospha-zenes; poly (ether-ester) copolymers, e.g. PEO-PLLA, or mixtures thereof; and biocompatible non-degrading materials, e.g. polydimethylsiloxane; poly (ethylene-vinylacetate); acrylate based polymers or coplymers, e.g. polybutylmethacrylate, poly (hydroxyethyl methyl-methacrylate); polyvinyl pyrrolidinone; fluorinated polymers such as polytetrafluoethylene; cellulose esters. When a polymeric matrix is used, it may comprise 2 layers, e.g. a base layer in which said inhibitor is incorporated, such as ethylene-co-vinylacetate and polybutylmethacrylate, and a top coat, such as polybutylmethacrylate, which acts as a diffusion-control of said inhibitor. Alternatively, said inhibitor may be comprised in the base layer and the adjunct may be incorporated in the outlayer, or vice versa.

Such biomaterial or medical delivery device may be biodegradable or may be made of metal or alloy, e. g. Ni and Ti, or another stable substance when intented for permanent use. The nutlin compound of the invention may also be entrapped into the metal of the stent or graft body which has been modified to contain micropores or channels. Also internal patches around the vascular tube, external patches around the vascular tube, or vascular cuff made of polymer or other biocompatible materials as disclosed above that contain the inhibitor of the invention may also be used for local delivery.

Said biomaterial or medical delivery device allow the nutlin compound releasing from said biomaterial or medical delivery device over time and entering the surrounding tissue. Said releasing may occur during 1 month to 1 year. The local delivery according to the present invention allows for high concentration of the nutlin compound of the invention at the disease site with low concentration of circulating compound. The amount of said nutlin compound used for such local delivery applications will vary depending on the compounds used, the condition to be treated and the desired effect. For purposes of the invention, a therapeutically effective amount will be administered.

The local administration of said biomaterial or medical delivery device preferably takes place at or near the vascular lesions sites. The administration may be by one or more of the following routes: via catheter or other intravascular delivery system, intranasally, intrabronchially, interperitoneally or eosophagal. Stents are commonly used as a tubular structure left inside the lumen of a duct to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to shear and disrupt the obstructions associated with the wall components of the vessel and to obtain an enlarged lumen.

The biomaterial of the invention may be coated with any other compounds as above described for pharmaceutical compositions.

The invention will be further illustrated by the following examples. However, these examples should not be interpreted in any way as limiting the scope of the present invention.

### EXAMPLE

### Materials and Methods

Methods:

### Mice

Wild-type mice aged 15-20 weeks were randomly allocated to room air or chronic hypoxia. Transgenic animals expressing the human 5-HTT gene in smooth muscle (SM22-5-HTT+ mice) were produced as previously described. All animal care and procedures were in accordance with institutional guidelines.

Nutlin-3 was administered at the increasing dosages of 6, 12, and 25 mg/Kg/day by intraperitoneal injection. At completion of treatment, lungs were removed and prepared for histological or Western blot analyses.

### Exposure to Chronic Hypoxia

Mice were exposed to chronic hypoxia (10% O₂) in a ventilated chamber (Biospherix, N.Y., USA). The hypoxic environment was established by flushing the chamber with a mixture of room air and nitrogen, and the gas was recirculated. The chamber environment was monitored using an oxygen analyzer. Carbon dioxide was removed by soda lime granules, and excess humidity was prevented by cooling of the recirculation circuit. Normoxic mice were kept in the same room, with the same light-dark cycle.

### Assessment of Pulmonary Hypertension

Mice exposed previously to hypoxia or room air or SM22-5-HTT+ mice were anaesthetized. After incision of the abdomen, a 26-gauge needle connected to a pressure transducer was inserted into the right ventricle through the diaphragm, and right ventricular systolic pressure (RVSP) was recorded immediately. Then, the thorax was opened and the lungs and heart were removed. The right ventricle (RV) was dissected from the left ventricle plus septum (LV+S), and these dissected samples were weighed for determination of Fulton's index (RV/LV+S). The lungs were fixed by intratracheal infusion of 4% aqueous buffered formalin. A midsagittal slice of the right lung was processed for paraffin embedding. Sections 5 μm in thickness were cut and stained with hematoxylin-phloxine-saffron for examination by light microscopy. In each mouse, a total of 20 to 30 intraacinar vessels accompanying either alveolar ducts or alveoli were examined by an observer who was blinded to the treatment or genotype. Each vessel was categorized as nonmuscular (no evidence of vessel wall muscularization), partially muscular (smooth muscle cells [SMCs] identifiable in less than three-fourths of the vessel circumference), or fully muscular (SMCs in more than three-fourths of the vessel circumference). The percentage of pulmonary vessels in each muscularization category was determined by dividing the number of vessels in that category by the total number counted in the relevant group of animals. For fully muscular vessels, video images were obtained and arterial diameters were measured using computerized image-analysis software. Percent wall thickness was then calculated as the diameter of the external elastic lamina minus the diameter of the internal lamina divided by the diameter of the external elastic lamina.

Studies on cultured human pulmonary artery smooth muscle cells (PA-SMC)

Cultured PA-SMCs were collected from pulmonary arteries of patients undergoing lung surgery for localized lung tumours. To determine the phenotypic characteristics of cultured PA-SMCs, the cells from each culture were assessed for expression of musclespecific contractile and cytoskeletal proteins, including smooth muscle cell α-actin and desmin.

To assess the effects of nutlin-3 on PASMC proliferation, cells were exposed to increasing concentration of nutlin-3a (2.5-10 μM) or vehicle in serum-free medium or in presence of PDGF-BB (50 ng/ml). After 48 hours, tetrazolium salt (MTT), (Sigma, Lyon, France) was added to each well (0.2 mg/ml). After 4 hours incubation at 37° C., the culture medium was removed and formazan crystals were solubilized by adding 500 μL of DMSO. Tetrazolium salt reduction to formazan within the cells was quantified by spectrophotometry at 520 nm.

To assess the effects of nutlin-3 on PASMC apoptosis, cells were trypsinized and resuspended in binding buffer then incubated with annexin V-FITC-conjugated antibody and stained with propidium iodide according to the manufacturer's instructions (Sigma-Aldrich). Annexin V staining and propidium iodide staining were detected by FACS (Becton Dickinson, Franklin Lakes, N.J., USA). Apoptotic cells were propidium iodide-positive cells and annexin V/propidium iodide-positive cells.

To assess the effects of nutlin on cell senescence, the percentage of beta-galactosidase (β-gap)-positive cells was measured after 48 hours incubation with nutlin-3 in presence or absence of vehicle or PDGF. The amounts of p53, p16, and p21 proteins or mRNA were determined by western blotting o by RT-qPCR, respectively.

Biological measurements in mice lungs and in cultured human pulmonary artery smooth muscle cells (PA-SMC)

p53, p16, p21, caspase and MDM2 proteins were detected and measured in lung tissues and/or cells using Western blotting. Levels of p21, Bax, and PUMA mRNA in lung tissues and/or cells were determined using RT-qPCR. Total mRNA was extracted from lung tissues and PA-SMC using RNeasy Mini Kit (Qiagen, ZA Courtaboeuf, France). First-strand cDNA was synthesized in reversed transcribed samples, as follows: 1 μg total RNA isolated from cells or lung tissues, 200 U/μL SuperScrip II RT (Invitrogen, Life Technologies, Cergy-Pontoise, France), 100 ng Random primers and 10 mM mixed dNTP. Quantitative PCR was performed in a 7900HT Real-Time PCR system (Applied Biosystems, ZA Courtaboeuf, France), using SYBR green Mix from Invitrogen.

### Chemicals and Drugs

Nutlin-3a was purchased from Bertin pharma (Montigny-le-Bretonneux, France).

Results:

Effects of treatment with Nutlin-3a on cultured human PA-SMC

Treatment of cultured human PA-SMCs by increasing doses of Nutlin-3 (2.5 to 10 μM) was associated with an activation of the p53 pathway, as evidenced by increased p53 protein and increased mRNA levels of the p53 target genes p21, Bax and PUMA. Treatment of PA-SMC with Nutlin-3 also induced a dose-dependent inhibition of cell proliferation stimulated by 0.2% FCS (Fetal Calf Serum) or 50 ng/ml PDGF, with an increased in the number of senescent cells manifested by an increase in the percentage of beta-galactosidase stained cells.

Effects of treatment with Nutlin-3a on pulmonary hypertension in mice

Nutlin-3 in dosages of 6 to 25 mg/Kg/day injected intraperitoneally to mice exposed to chronic hypoxia prevented pulmonary hypertension, right ventricular hypertrophy, and distal pulmonary artery muscularization. Treatment with 12 mg/Kg/day of Nutlin-3 also partially reversed PH in SM22-5-HTT+ mice. In both hypoxic and SM22-5-HTT+ mice, Nutlin-3 treatment was associated with marked increases in lung p53 protein, p21 mRNA, and p21 protein. In contrast, pulmonary hypertension induced by chronic hypoxia in p53-deficient (p53^{-/-}) mice which was of similar severity as wild-type mice, remained unaffected by treatment with nutlin-3a. Thus, the effects mediated by Nutlin required increased expression of p53 in the lung, as indicated by the inability of Nutlin-3 to prevent chronic hypoxia-induced PH in p53−/− mice. The protective effects of Nutlin-3 were associated with a simultaneous increase in apoptosis and decrease in PA-SMC proliferation.

Conclusion:

Nutlin-3 mitigates established PH by activating the p53 pathway.

### References

Throughout this application, various references describe the state of the art to which this invention pertains. The disclosures of these references are hereby incorporated by reference into the present disclosure.

Deng J, Dayam R, Neamati N. Patented small molecule inhibitors of p53-MDM2 interaction. Expert Opin Ther Pat. 2006; 16(2): 165-88.

Fukumoto Y, Shimokawa H. Recent Progress in the Management of Pulmonary Hypertension. Circ J. 2011.

Fuso L, Baldi F, Di Perna A. Therapeutic strategies in pulmonary hypertension. Front Pharmacol. 2011; 2: 21.

Klein C, Vassilev L T. Targeting the p53-MDM2 interaction to treat cancer. Br J Cancer. 2004; 91(8): 1415-9.

Raiesdana A, Loscalzo J. Pulmonary arterial hypertension. Ann Med. 2006; 38(2): 95-110.

Simonneau G, Galiè N, Rubin L J, Langleben D, Seeger W, Domenighetti G, Gibbs S, Lebrec D, Speich R, Beghetti M, Rich S, Fishman A. Clinical classification of pulmonary hypertension. J Am Coll Cardiol. 2004; 43(12 Suppl S):5S-12S.

Vassilev L T, Vu B T, Graves B, Carvajal D, Podlaski F, Filipovic Z, Kong N, Kammlott U, Lukacs C, Klein C, Fotouhi N, Liu E A. In vivo activation of the p53 pathway by small-molecule antagonists of MDM2. Science 2004; 303 (5659): 844-848.

Vassilev LT. p53 Activation by small molecules: application in oncology. J Med Chem. 2005; 48(14): 4491-9.

1-6. (canceled)

7. A pharmaceutical composition comprising
a nutlin compound and a pharmaceutically acceptable carrier.

8. A Kit comprising.
a first pharmaceutical composition comprising a nutlin compound and a pharmaceutically acceptable carrier and
a second pharmaceutical composition comprising a one or more phosphodiesterase (PDE) inhibitors selected from the group consisting of a PDE3 inhibitor, a PDE4 inhibitor, a PDE5 inhibitor and a pharmaceutically acceptable carrier.

9. The pharmaceutical composition of claim 7, wherein said nutlin compound is a nutlin analogue selected in the group consisting of a cis-imidazolin analogue, nutlin 1, nutlin 2, nutlin 3 and nutlin 3a.

10. A method of preventing or treating pulmonary hypertension in a subject in need thereof, comprising
administering to said subject a therapeutically effective amount of a nutlin compound.

11. The method of claim 10, wherein said subject is afflicted with HIV infection.

12. The method of claim 10, wherein said nutlin compound is an analogue is selected from the group consisting of a cis-imidazolin analogue, nutlin 1, nutlin 2, nutlin 3 and nutlin 3a.

13. A method for inducing pulmonary-artery smooth muscle cell senescence or apoptosis and/or inhibiting pulmonary-artery smooth muscle cell proliferation in a subject of need thereof, comprising
administering to said subject a therapeutically effective amount of a nutlin compound.

14. The method of claim 13, wherein said subject is afflicted with HIV infection.

15. The method of claim 13, wherein said nutlin compound is a nutlin analogue is selected from the group consisting of a cis-imidazolin analogue, nutlin 1, nutlin 2, nutlin 3 and nutlin 3a.

16. The method of claim 13, wherein said step of administering is performed by providing said subject with a biomaterial or medical delivery device comprising said nutlin compound.

17. A biomaterial or medical delivery device comprising a nutlin compound.

18. The biomaterial or medical delivery device of claim 17, wherein said nutlin compound is coated on said biomaterial or medical delivery device.

19. The biomaterial or medical delivery device of claim 17, wherein said nutlin compound is a nutlin analogue is selected from the group consisting of a cis-imidazolin analogue, nutlin 1, nutlin 2, nutlin 3 and nutlin 3a.

20. The biomaterial or medical delivery device of claim 18, wherein said biomaterial or medical delivery device is selected from the group consisting of: an endovascular prostheses, a bypass grafts, an internal patch for placement around the vascular tube, an external patch for placement around the vascular tube, a vascular cuff, and an angioplasty catheter.

21. The biomaterial or medical delivery device of claim 20, wherein said endovascular prostheses is a stent.
